(19) United States
(12) Patent Application Publication
(10) Pub. No.: US 2018/0184986 A1
(43) Pub. Date: Jul. 5, 2018

(54) ELECTRIC PERMITTIVITY AND MAGNETIC PERMEABILITY BIOSENSING SYSTEM

(71) Applicant: Wichita State University, Wichita, KS (US)

(72) Inventors: Kim Cluff, Wichita, KS (US); Jeremy Patterson, Wichita, KS (US)

(73) Assignee: Wichita State University, Wichita, KS (US)

(21) Appl. No.: 15/738,505

(22) PCT Filed: May 3, 2016
(86) PCT No.: PCT/US2016/030583

§ 371 Date: Dec. 20, 2017

Related U.S. Application Data
(60) Provisional application No. 62/185,198, filed on Jun. 26, 2015.

(51) Int. Cl. A61B 5/00; A61B 5/053; A61B 5/0265; A61B 5/0452; A61B 5/087; A61B 5/03; A61B 5/0476; A61B 5/103

(52) U.S. Cl. CPC A61B 5/7445; A61B 5/002; A61B 5/6804; A61B 5/053; A61B 5/0265; A61B 2560/0412; A61B 5/087; A61B 5/4851; A61B 5/031; A61B 5/0476; A61B 5/1036; A61B 5/0452

(57) ABSTRACT

A biosensor system including a biosensor patch and an interrogation device. The biosensor patch includes an open circuit self-resonating pattern for passively generating a response signal when excited by an interrogation signal. The response signal may have a frequency amplitude, phase, and bandwidth that may change according to an electro-magnetic response of a biological component of a user. The interrogation device transmits an interrogation signal to the biosensor patch and receives the response signal in return. The interrogation device may run an application for decoding and analyzing the response signal to generate data corresponding to the health of the biological component.

### RELATED APPLICATIONS

This non-provisional patent application claims priority benefit with regard to all common subject matter of earlier-filed U.S. Provisional Patent Application Ser. No. 62/185,198 filed on Jun. 26, 2015 and entitled “ELECTRIC PERMITTIVITY AND MAGNETIC PERMEABILITY BIOSENSING SYSTEM”. The identified earlier-filed provisional patent application is hereby incorporated by reference in its entirety into the present application.

### FEDERALLY SPONSORED RESEARCH OR DEVELOPMENT

This invention was made with government support under Grant #T4-6500-WSU awarded by the National Institute of Aerospace (NIA) (NASA PRIME).

### BACKGROUND

Biosensor systems are used to monitor or detect biological processes, functions, or statuses, which can be correlated with signs and/or symptoms of overall health and used as indicators of health-related issues to improve and maintain a user's health. Biosensor systems may include biosensors configured to detect electrical signals, optical absorption, magnetic signals, acceleration, or pressure changes in or on a biological system. However, conventional biosensors typically include complex circuitry, such as operational amplifiers, capacitors, resistors, transistors—all of which can fail and act as a limiting factor to continuous reliability. Further limiting factors of conventional biosensing systems are the need to connect to an external power source, which limits the mobility of the user, or include a portable battery, which increases the risk that the biosensor loses power and fails to detect signs of health issues. Finally, in some cases the biosensing signals are not easily interpretable by the general public, such as in electrocardiogram (ECG) waveforms and pulse volume recording (PVR) waveforms. Interpretation of ECG and PVR waveforms requires specialized training, advanced knowledge of the biological system, and knowledge of the electrical system. Thus, there is a need for a robust smart biosensor (without electrical component limiting factors) to acquire, synthesize, interpret, and communicate results in a simple and familiar manner.

Further limitations in current biofluid measuring biosensors are of particular interest to this disclosure. Typical wearable biosensing systems, e.g., for measuring biofluid hemodynamics (blood flow, blood perfusion, blood oxygen concentration, or blood pulse), include optical IR photo-sensitive sensors. Theses biosensors are not able to detect blood flow in deep arteries and are limited to the peripheries such as the hands, fingers, toes, or arteries that have a palpable pulse, and are superficial at best. Their main limitation is the penetration depth of optical based IR photosensitive sensors, such as in pulse oximetry. For example, photon migration is limited to the optical transport mean free path and attenuates quickly at 1 mm as defined by the optical diffusion limit.

### SUMMARY

Embodiments of the present invention solve the above problems and provide a distinct advance in the art of biosensor systems.

One embodiment of the present invention is a biosensor system including a biosensor patch and an interrogation device. The biosensor patch may include an electrically-conductive material in an open circuit self-resonating pattern for passively generating a response signal when excited by an interrogation signal. The response signal may have a frequency that may change according to an electro-magnetic response such as a change in electric permittivity, magnetic permeability, and/or electric conductivity of a biological component such as a biofluid, blood, heart, lung, brain, bone, or even muscle of a user. The interrogation device may include a transceiver, a processor, a memory, and a display. The transceiver may transmit an interrogation signal to the biosensor patch and may receive the response signal from the biosensor patch. The processor may run an application for decoding and analyzing the response signal so as to generate data corresponding to the health of the user's biological component. The application may have a graphical user interface including virtual controls for receiving user input. The application may indicate when the data signifies a symptom or health issue. The memory may store the data thereon and the display screen may display the graphical user interface (GUI) and the graphical elements representative of the data for ease of understanding the data and manipulating the biosensor system.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is an environmental view of a biosensor system constructed in accordance with an embodiment of the present invention;

FIG. 2 is an environmental view of a biosensor patch of the biosensor system of FIG. 1 and magnetic fields generated by the biosensor patch interacting with bloodflow of a user's arm;

FIG. 3 is an environmental view of a biosensor patch of the biosensor system of FIG. 1 being worn on a user's leg;

FIG. 4 is an environmental view of a biosensor patch of the biosensor system of FIG. 1 embedded in a garment;

FIG. 5 is an environmental view of a biosensor patch of the biosensor system of FIG. 1 screen printed on a garment;

FIG. 6 is a schematic view of an interrogation device of the biosensor system of FIG. 1;

FIG. 7 is a schematic view of the biosensor system of FIG. 1; and

FIG. 8 is an environmental view of the biosensor patch of FIG. 1 being worn near a user's heart.

The drawing figures do not limit the present invention to the specific embodiments disclosed and described herein. The drawings are not necessarily to scale, emphasis instead being placed upon clearly illustrating the principles of the invention.

### DETAILED DESCRIPTION OF THE EMBODIMENTS

The following detailed description of embodiments of the present invention is intended to describe aspects of the invention in sufficient detail to enable those skilled in the art to practice the invention. Other embodiments can be utilized and changes can be made without departing from the scope of the invention. The following detailed description is, therefore, not to be taken in a limiting sense. The scope of the present invention is defined only by claims presented in subsequent regular utility applications, along with the full scope of equivalents to which such claims are entitled.

In this description, references to “one embodiment,” “an embodiment,” or “embodiments” mean that the feature or features being referred to are included in at least one embodiment of the technology. Separate references to “one embodiment,” “an embodiment,” or “embodiments in this description do not necessarily refer to the same embodiment and are also not mutually exclusive unless so stated and/or except as will be readily apparent to those skilled in the art from the description. For example, a feature, step, etc. described in one embodiment may also be included in other embodiments, but is not necessarily included. Thus, the present technology can include a variety of combinations and/or integrations of the embodiments described herein.

Embodiments of the present invention solve various problems, including those described in the background section above, and provide a distinct advance in the art of biosensor systems. More particularly, embodiments of the present invention provide a biosensor system for monitoring fluid and tissue health in biological systems by detecting changes in electric permittivity, magnetic permeability, electric conductivity, and/or other electromagnetic response of components of the biological systems. For instance, the biosensor patch and system can be used to monitor arterial blood flow, pulse volume recordings, peak reactive hyperemia, heart activity, brain activity, intracranial pressure, tissue proximity, muscle tissue health, changes in bone density, and lung intrapleural space health via an interrogation signal.

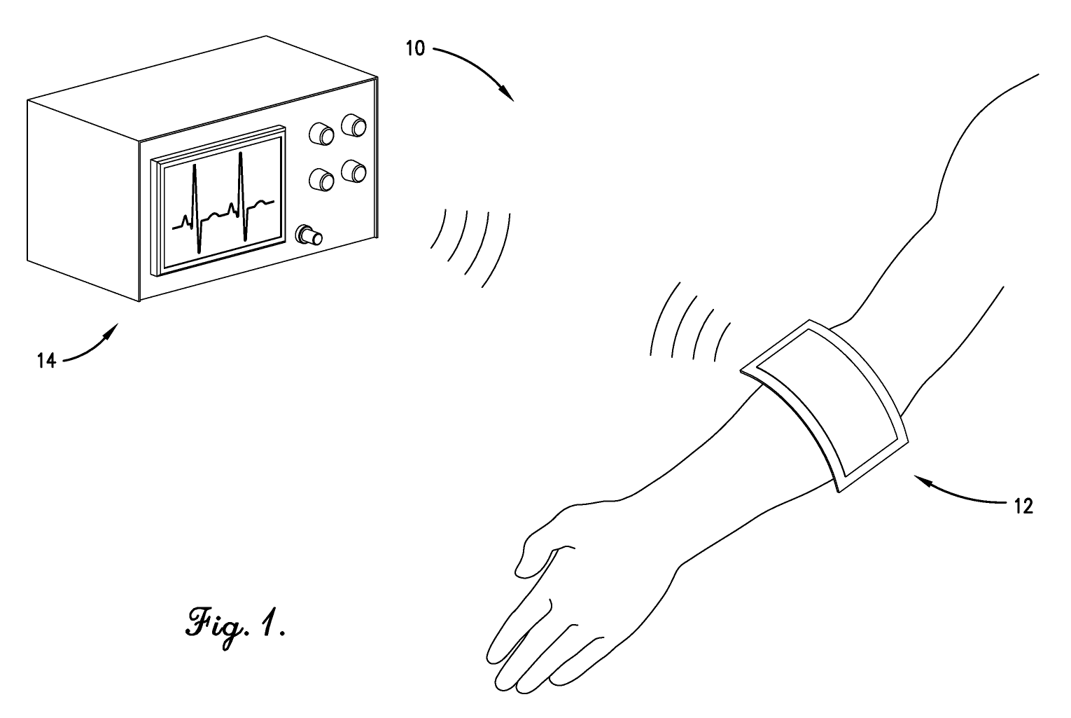

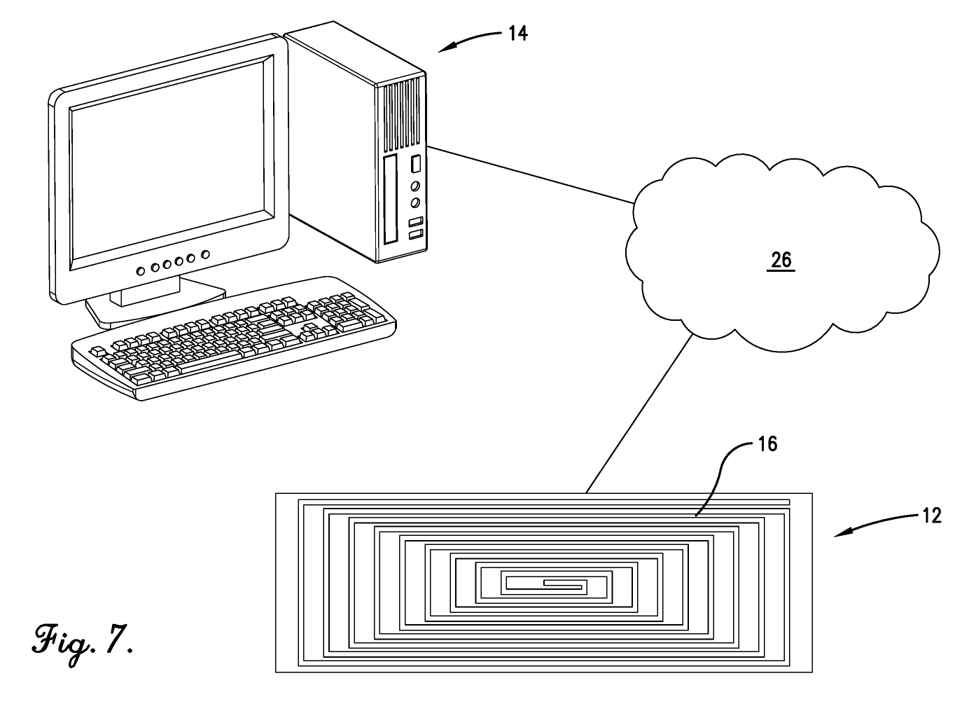

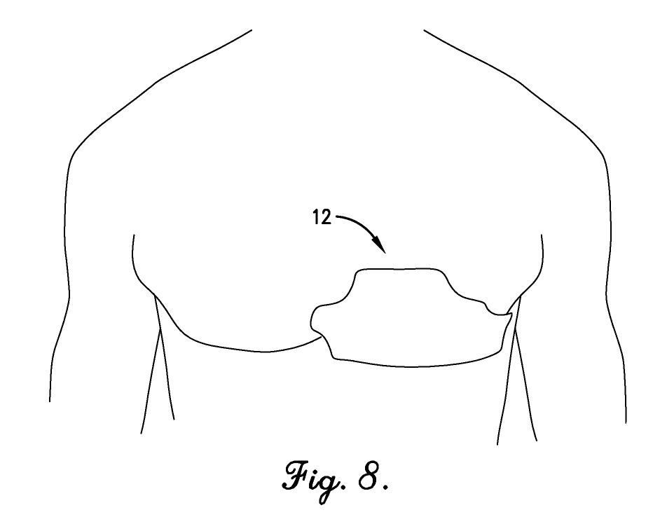

Turning to FIGS. 1-8, a first embodiment of the present invention is a biosensor system 10 that includes a biosensor patch 12 and an interrogation device 14.

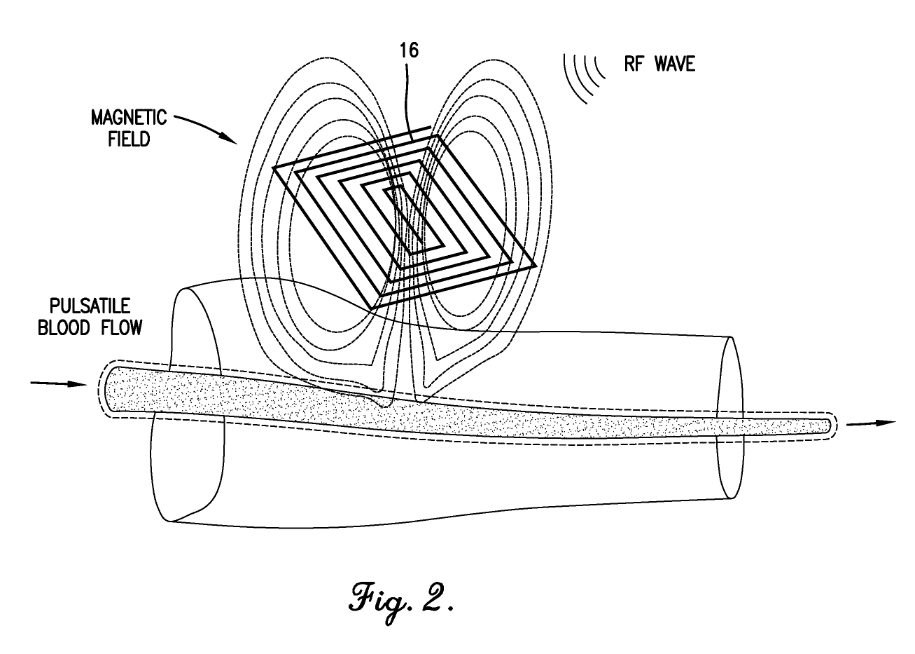

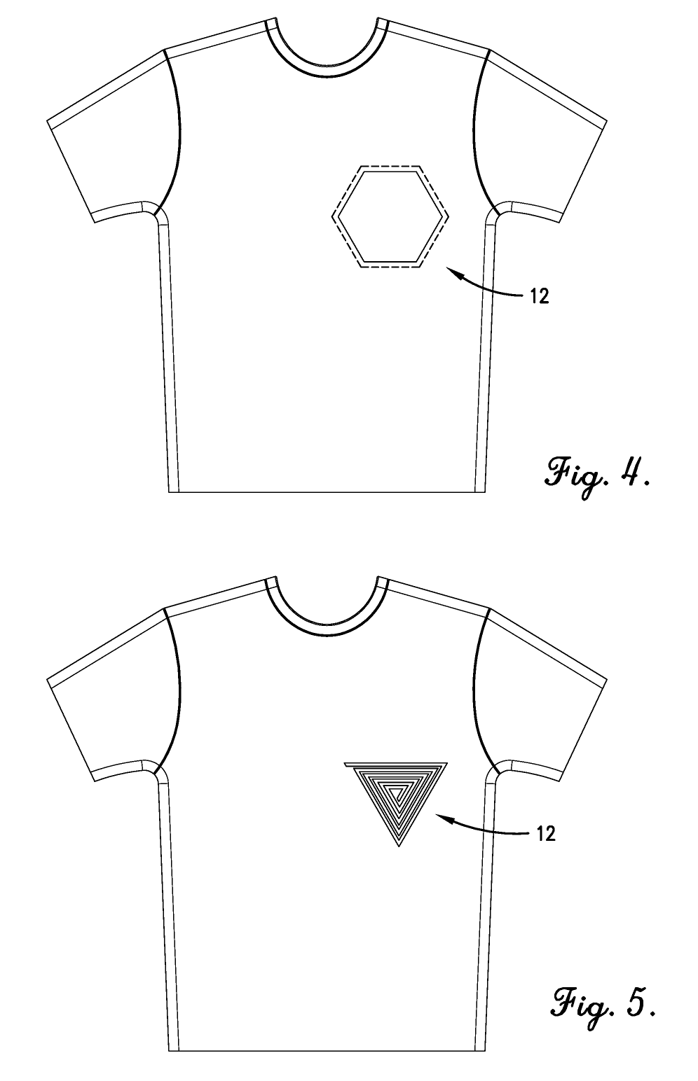

The biosensor patch 12 passively generates a response signal when excited by an interrogation signal and includes a wireless open circuit self-resonating spiral 16 (without electrical components) or similar wireless open circuit self-resonating pattern. The spiral 16 may be planar, square, rectangular, triangular, hexagonal, octagonal, circular, oblong, or any other shape or configuration, as shown in FIGS. 2-5. The spiral 16 may be a micro-coil, array of micro-coils, or layers of micro-coils. The micro-coil arrays can work together as a network and/or be configured to monitor various frequency responses of various applications in a biological system as described below. The biosensor patch 12 may be disposable and/or reusable and may include an adhesive, elastic band, strap, or similar fastener for removably attaching and/or securing the biosensor patch 12 to a user's skin or clothing. The biosensor patch 12 may also be embedded in (FIG. 4), screen printed on (FIG. 5), or stitched on the user's skin or clothing, or otherwise attached to the user or the user's clothing. In one embodiment, the biosensor patch 12 may be a square planar spiral sensor. SansEC (Sans Electrical Connection) sensors are exemplary types of sensors for use in the invention. SansEC sensors are self-resonating patterns of electrically-conductive material that do not use electrical connections. Exposing the sensor to oscillating electromagnetic fields induces an electromotive force in the pattern. When the pattern is electrically active, it responds with its own harmonic magnetic and electric fields. The amplitude and resonant frequency response of the sensor pattern will change as a function of the electric permittivity and permeability of the environment that the sensor is exposed to. Exemplary electrically-conductive materials for forming the spiral pattern include copper, silver, and the like. The spiral patterns could also be formed from a biomaterial polymer doped with electrically-conductive particles and 3D printed. In another embodiment the planar spiral is sewn or screen-printed into close fitting garments using conductive threads or ink of the above-mentioned materials.

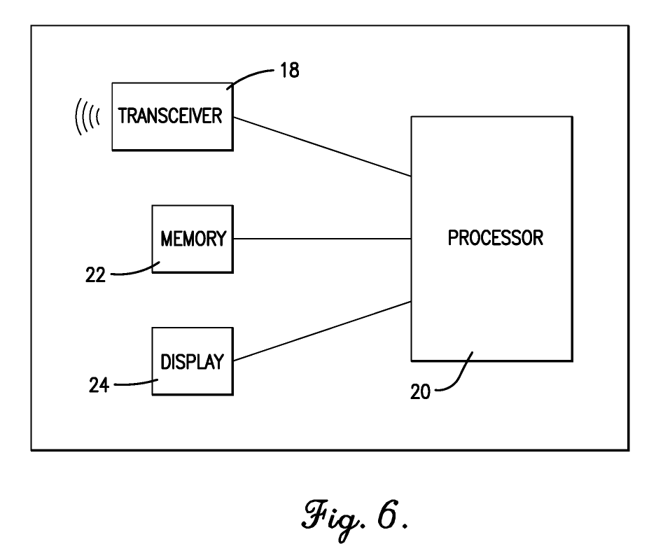

The interrogation device 14 generates an interrogation signal for exciting the biosensor patch 12 and analyzes the response signal(s) passively generated by the biosensor patch 12. The interrogation device 14 may include a transceiver 18, processor 20, memory 22, and a display 24, as shown in FIG. 6. The interrogation device 14 may be a vector network analyzer (VNA), as shown in FIG. 1, smartphone, smartwatch, personal digital assistant (PDA), tablet, laptop, gaming device, desktop computer, or any other suitable portable or non-portable computing device capable of transmitting and receiving the signal.

The transceiver 18 transmits the interrogation signal to the biosensor patch 12 and receives the response signal from the biosensor patch 12 directly via near field communication (NFC) or other similar direct wireless transmission or over a wireless communication network 26. The transceiver 18 may be an antenna connectable to the interrogation device 14 or integrated with the interrogation device 14. The transceiver 18 may transmit an NFC signal or other similar interrogation signal.

The wireless communication network 26 may be the Internet or any other wireless communication network such as a local area network, a wide area network, or an intranet. The wireless communication network 26 may include or be in communication with a network capable of supporting wireless communications such as the wireless networks operated by AT&T, Verizon, or Sprint. The wireless communication network 26 may also be combined or implemented with several different networks.

The processor 20 decodes and analyzes the response signal via an application or computer program as described below. The processor 20 may comprise any number and combination of processors, controllers, integrated circuits, programmable logic devices, or other data and signal processing devices for carrying out the functions described herein, and may additionally comprise one or more memory storage devices, transceivers, receivers, and/or communication busses for communicating with the various devices of the system. In various embodiments of the invention, the computing devices may comprise a memory element, a communication component, a display, and/or a user interface.

In embodiments of the invention, the processor 20 may implement an application or computer program to perform some of the functions described herein. The application may comprise a listing of executable instructions for implementing logical functions in the mobile device. The application (described in more detail below) can be embodied in any computer readable medium for use by or in connection with an instruction execution system, apparatus, or device, and execute the instructions including the mobile device. The various actions and calculations described herein as being performed by or using the application may actually be performed by one or more computers, processors, or other computational devices, independently or cooperatively executing portions of the application.

The memory 22 may be any computer readable medium that can contain, store, communicate, propagate, or transport the application for use by or in connection with the instruction execution system, apparatus, or device. The computer readable medium can be, for example, but not limited to, an electronic, magnetic, optical, electro magnetic, infrared, or semi conductor system, apparatus, device or propagation medium. More specific, although not inclusive, examples of the computer readable medium would include the following: a portable computer diskette, a random access memory (RAM), a read only memory (ROM), an erasable, programmable, read only memory (EPROM or flash memory), and a portable compact disk read only memory (CDROM), and combinations thereof.

The application allows the user or a caregiver to easily check vital signs or check for medical issues of the user and will now be described in more detail. The application may generate a graphical user interface (GUI) having virtual buttons, text boxes, and other controls for entering text and other inputs and for changing settings and other parameters. The application may display graphic windows and/or displays for displaying raw data such as frequency response(s) to changes in electrical permittivity, magnetic permeability, and/or electric conductivity in graphical or tabular form. The application may also display contextual data such as heartrate, heartbeat, blood pressure, intrapleural gas concentrations, and other contextual data in a format understandable by non-medical individuals. The application may also display prompts indicating a health issue has been detected or that further medical care should be obtained, as described below.

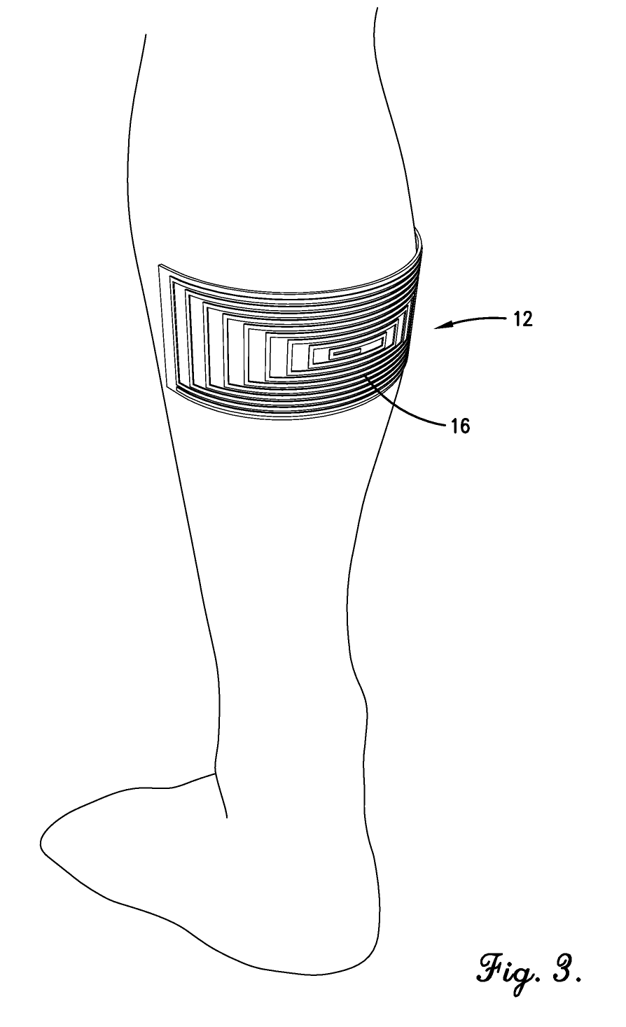

Turning again to FIGS. 1-8 use of the biosensor system 10 will now be described in more detail. The user may place the biosensor patch 12 on the user's skin near the user's heart, on the user's arm or leg, or near any other location of interest. For example, the user may place the biosensor patch 12 above the user's calf for detecting reduced blood flow due to atherosclerotic blockage, as shown in FIG. 3. The user may then initiate a reading by selecting an option from the GUI to begin interrogation. The processor 20 may then instruct the transceiver 18 to transmit an interrogation signal to the biosensor patch 12 via NFC or similar technology. The interrogation signal may then excite the self-resonating spiral 16 so that the self-resonating spiral 16 passively generates a response signal. Characteristics of the response signal may change according to changes in electric permittivity, magnetic permeability, and/or electric conductivity of the underlying biological feature. For example, the frequency of the response signal may change in correspondence with systole and diastole periods of the user's circulatory system. A pattern of the frequency over time may then match the user's pulsatile blood flow pattern. The application may then detect blockages, blood flow changes, pulse volume recordings, high or low blood pressure, and/or other medical issues based on these correlations, as described in more detail below. The application may alert the user or the caregiver to these medical issues so that the caregiver may then administer treatment accordingly.

In one embodiment, the biosensor system 10 may be used to monitor blood flow or generate pulse volume recordings for screening for peripheral artery disease (PAD). The biosensor system 10 may be used for early detection and post-surgical treatment monitoring of blood flow (limb hemodynamics) by detecting changes in electrical permittivity and/or magnetic permeability of the blood as it flows.

In another embodiment, the biosensor system 10 may be used to monitor heart issues such as symptoms associated with sudden cardiac death (hypertrophic cardiomyopathy). The biosensor patch 12 may be placed near the user's heart to monitor conduction and/or blood flow so as to screen for sudden cardiac death arrhythmias, as shown in FIG. 8.

In another embodiment, the biosensor system 10 may be used to monitor muscle tissue health in PAD patients. The biosensor system 10 may detect changes in electric permeability, magnetic permittivity, and/or electric conductivity of the muscle due to injury or other ailments. This may also be effective in detecting or preempting injuries such as foot sores in individuals with diabetes.

In yet another embodiment, the biosensor system 10 may be configured to detect important changes in electric permittivity, magnetic permeability, and/or electric conductivity of cancerous cells and used as a diagnostic for skin melanoma, cancer biopsy margin measurements, or other cancer cell detection.

The biosensor system 10 may also be used to monitor lung damage via air flow detection (e.g., fluid flow in the plural space). For example, the biosensor patch 12 may be attached to a chest tube to detect CO₂ or other inhaled foreign substance such as helium.

In still another embodiment, the biosensor system 10 may be woven, built, or otherwise integrated into prosthetic implants. For example, the biosensor system 10 may be built or etched into a hip prosthesis allowing real-time measures of pressures and data concerning healing. Many individuals undergoing these procedures have a metal sensitivity and may have a toxic reaction to the material in the prosthetic implant. Data from the prosthesis would allow for a rapid detection of infection and prevent tissue necrosis.

In another embodiment, the biosensor system 10 may be configured to detect important changes in electric permittivity, magnetic permeability, and/or electric conductivity of infection. Patients receiving surgical implantation devices such as artificial hips, knees, or other implantable devices are often at risk of infection.

In yet another embodiment, the biosensor system 10 may be used to monitor brain activity. For example, the biosensor system 10 may be used to measure fluid level (brain swelling/intracranial pressure) and electrical activity in the brain for detecting or monitoring head concussions, shaking baby syndrome, pharmacological effects, cognitive fatigue, and other brain related ailments.

Although each of the above listed examples detail a detection of a health problem, it should be noted that the biosensor system may be used to identify health improvements or maintenance. In one embodiment, the biosensor 10 may be used to detect post-surgery healing rate, for example. The biosensor system 10 may be built into garments to detect acute and chronic physiological changes from human activity and stress. Additionally, the biosensor system 10 may be used to measure contact pressure. For example, the biosensor system 10 may be imbedded into shoes to measure foot pressure while walking, or into gloves to measure grip strength and fatigue.

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. A biosensor system comprising:
a biosensor patch comprising an electrically-conductive material in an open circuit self-resonating pattern configured to passively generate a response signal when excited by an interrogation signal, the response signal having a frequency, amplitude, phase, and bandwidth that may change according to an electro-magnetic response of a biological component of a user; and
an interrogation device including:
a transceiver configured to transmit an interrogation signal to the biosensor patch and receive the response signal generated by the biosensor patch;
a processor configured run an application for decoding and analyzing the response signal to generate data corresponding to the health of the biological component, the application having a graphical user interface including virtual controls for receiving user input, the application being configured to indicate when the data signifies a symptom or a health issue;
a non-transitory computer readable memory for storing the application and the data thereon; and
a display screen configured to display the graphical user interface and graphical elements representative of the data.

2. The biosensor system of claim 1, wherein the transceiver of the interrogation device is configured to transmit the interrogation signal to the biosensor patch and receive the response signal generated by the biosensor patch via near field communication (NFC) technology.

3. The biosensor system of claim 1, wherein the biosensor patch is configured to generate a response signal having a frequency, amplitude, phase, and bandwidth that may change according to electric permittivity of a biological component of the user.

4. The biosensor system of claim 1, wherein the biosensor patch is configured to generate a response signal having a frequency, amplitude, phase, and bandwidth that may change according to magnetic permeability of a biological component of the user.

5. The biosensor system of claim 1, wherein the biosensor patch is configured to generate a response signal having a frequency that may change according to electric conductivity of a biological component of the user.

6. The biosensor system of claim 1, wherein the biosensor patch is configured to be worn on a wearer's skin.

7. The biosensor system of claim 6, wherein the biosensor patch further includes a fastener for securing the biosensor patch on the wearer's skin.

8. The biosensor system of claim 1, wherein the biosensor patch is configured to be secured to or embedded in a wearer's clothing.

9. The biosensor system of claim 1, wherein the biosensor patch is configured to be attached to a medical device.

10. The biosensor system of claim 1, wherein the biosensor patch is configured to be integrated into a medical implant.

11. The biosensor system of claim 1, wherein the self-resonating pattern comprises a planar spiral.

12. The biosensor system of claim 1, wherein the electrically-conductive material in an open circuit self-resonating pattern is part of a networked array of electrically-conductive material in respective open circuit self-resonating patterns.

13. The biosensor system of claim 1, wherein the application includes a graphical user interface (GUI) for displaying data corresponding to the frequency, amplitude, phase, and bandwidth of the response signal in a medical format.

14. The biosensor system of claim 1, wherein the application includes a graphical user interface (GUI) for displaying data corresponding to the frequency, amplitude, phase, and bandwidth of the response signal in a format understandable by non-medical individuals.

15. The biosensor system of claim 1, wherein the application is configured to generate a prompt indicating that a health issue has been detected based on a frequency, amplitude, phase, and bandwidth of the response signal.

16. The biosensor system of claim 1, wherein the application is configured to display a pattern of the frequency of the response signal over time.

17. A biosensor system comprising:
a biosensor patch comprising an electrically-conductive material in an open circuit self-resonating spiral configured to passively generate a response signal when excited by an interrogation signal, the response signal having a frequency, amplitude, phase, and bandwidth that may change according to electric permittivity, magnetic permeability, and/or electric conductivity of a biological component of a user, the biosensor patch being configured to be worn on the user's skin; and
an interrogation device including:
a transceiver configured to transmit an interrogation signal to the biosensor patch and receive the response signal generated by the biosensor patch via NFC technology;
a processor configured run an application for decoding and analyzing the response signal to generate data corresponding to the health of the biological components, the application having a graphical user interface including virtual controls for initiating transmission of an interrogation signal, the application being configured to indicate when the data signifies a symptom or a health issue;
a non-transitory computer-readable memory for storing the application and the data thereon; and
a display screen configured to display the graphical user interface and graphical elements representative of the data.
